(19) United States
(12) Patent Application Publication
(10) Pub. No.: US 2016/0091494 A1
(43) Pub. Date: Mar. 31, 2016

(54) DIAGNOSTIC METHOD FOR HEPATIC CANCER

(71) Applicant: IMPERIAL INNOVATIONS LTD, London (GB)

(72) Inventors: Simon TAYLOR-ROBINSON, London (GB); Elaine HOLMES, Surrey (GB); Nimzing LADEP, London (GB); Anthony DONA, London (GB)

(21) Appl. No.: 14/785,976

(22) PCT Filed: Apr. 22, 2014
(86) PCT No.: PCT/GB2014/051249

§ 371 Date: Oct. 21, 2015

(30) Foreign Application Priority Data
Apr. 22, 2013 (GB) 1307256.6

(51) Int. Cl. G01N 33/574

(52) U.S. Cl. CPC G01N 33/57438

(57) ABSTRACT

Methods and kits for analysing a sample from a test subject. The methods involve determining the level of at least one compound selected from the group consisting of N-acetylglutamate, methionine, acetylcarnitine, indole-3-acetate, 2-oxoglutarate, anserine, aspartate and butyrate in the sample from the test subject; and comparing the level of the at least one compound determined to at least one control level, wherein the levels of the at least one compound are indicative of whether the subject has hepatic cancer.

### TECHNICAL FIELD

This invention relates to diagnostic methods for identifying subjects suffering from hepatic cancer.

### BACKGROUND OF THE INVENTION

Hepatic cancer may take the form of primary hepatic cancer which is considered to be cancer which originates from the liver, or secondary hepatic cancer where the cancer originates in another organ and spreads to the liver. Hepatocellular carcinoma (HCC) is the most common form of primary hepatic cancer and is the third most common cause of cancer death worldwide^{1,2,3}. The disease is particularly prevalent in the developing world, and especially sub-Saharan Africa and Asia⁴, where several countries display a high incidence of over 20 cases for every 100,000 people. If the cancer cannot be completely removed, the disease is usually fatal within 3-6 months⁵. Symptoms of HCC can be very severe and include jaundice, bloating from ascites, easy bruising from blood clotting abnormalities, loss of appetite, unintentional weight loss, abdominal pain, especially in the upper-right part, nausea, emesis, and fatigue⁶.

Current methods of diagnosis include screening for HCC using serum α-fetoprotein (AFP), a fetal glycoprotein that is normally undetectable soon after birth. Most HCC secrete AFP, but about 30% do not and AFP has poor sensitivity and specificity of less than 70%^{7,8,9,10}. Furthermore, AFP testing of serum can be prohibitively expensive and therefore unavailable in parts of Africa and Asia. Many other serum markers including des-gamma-carboxyprothrombin, anti-p53, gamma-glutamyl-transpeptidase and isoferritin are also used in screening for HCC, but are more expensive and like AFP display a low degree of sensitivity and specificity^{11,12}.

The problem of sensitivity and specificity of serum markers for HCC is further heightened by the fact that patients suffering with cirrhosis can be difficult to distinguish from patients suffering from HCC using current methods. Early diagnosis of HCC is very important, since lesions below 2 cm are curable with resection or transplant. There is therefore a real need in the art for the development of a test with a level of sensitivity and specificity high enough to distinguish between patients with HCC, cirrhosis and healthy controls, even at early stages of disease.

In addition to the problems involving low sensitivity and specificity of serum markers, the areas where the disease is most prevalent give rise to ethical considerations. In many African and Asian countries, religious beliefs prohibit the use of invasive techniques in screening for disease. It is also important that information from the test be available quickly after testing is carried out since patients in the developing world may live many days journey from the health clinic.

HCC can be diagnosed more accurately using CT scans, MRI scans and biopsy¹³. However, the costs and time involved with these techniques as well as the invasive nature of biopsy, mean that these tests are not suitable for developing areas where the disease displays the highest prevalence.

There is a need for the development of a method of diagnosing HCC which is both specific and sensitive in all test subjects, as well as being practical and available for use in both the developed and the developing world. A sensitive and specific test would allow for detection of the disease at an early stage, leading to a significant improvement in the prognosis for HCC sufferers.

### DISCLOSURE OF THE INVENTION

Accordingly, the invention provides a method for analysing a sample from a test subject comprising:
i) determining the level of at least one compound selected from the group consisting of N-acetylglutamate, methionine, acetylcarnitine, indole-3-acetate, 2-oxoglutarate, anserine, aspartate and butyrate in the sample from the test subject; and
ii) comparing the level of the at least one compound determined in step i) to at least one control level, wherein the levels of the at least one compound are indicative of whether the subject has hepatic cancer.

Surprisingly, the inventors have found that the levels of N-acetylglutamate, methionine, acetylcarnitine, indole-3-acetate, 2-oxoglutarate, anserine, aspartate and butyrate are significantly increased in samples taken from subjects with hepatic cancer compared with samples taken from subjects which do not have hepatic cancer. This contrasts with previous markers (glycine, trimethylamine-N-oxide, hippurate and citrate) which have been found to be decreased in HCC subjects compared to control subjects. The method of the invention can be used as a diagnostic method to determine whether a test subject has hepatic cancer. The levels of these compounds have never before been measured as part of a method for diagnosing hepatic cancer. The measurement of the levels of these particular compounds allows for sensitive and specific screening for hepatic cancer, and the fact that the compounds may be measured using a wide range of simple assays will aid diagnosis for hepatic cancer in the developing world where the disease is most prevalent.

The method of the invention may comprise i) determining the level of N-acetylglutamate in a sample from a test subject; and comparing the level of N-acetylglutamate determined in step i) to a control level.

The method of the invention may comprise i) determining the level of methionine in a sample from a test subject; and comparing the level of methionine determined in step i) to a control level.

The method of the invention may comprise i) determining the level of acetylcarnitine in a sample from a test subject; and comparing the level of acetylcarnitine determined in step i) to a control level.

The method of the invention may comprise i) determining the level of indole-3-acetate in a sample from a test subject; and comparing the level of indole-3-acetate determined in step i) to a control level.

The method of the invention may comprise i) determining the level of 2-oxoglutarate in a sample from a test subject; and comparing the level of 2-oxoglutarate determined in step i) to a control level.

The method of the invention may comprise i) determining the level of anserine in a sample from a test subject; and comparing the level of anserine determined in step i) to a control level.

The method of the invention may comprise i) determining the level of aspartate in a sample from a test subject; and comparing the level of aspartate determined in step i) to a control level.

The method of the invention may comprise i) determining the level of butyrate in a sample from a test subject; and comparing the level of butyrate determined in step i) to a control level.

The method of the invention may comprise determining the level of at least two compounds selected from the group consisting of N-acetylglutamate, methionine, acetylcarnitine, indole-3-acetate, 2-oxoglutarate, anserine, aspartate and butyrate and comparing the level of the at least two compounds to a control level. In one embodiment, the method may comprise determining the level of N-acetylglutamate and methionine and comparing the levels to control levels. In another embodiment the method may comprise determining the level of N-acetylglutamate and acetylcarnitine and comparing the levels to control levels. In another embodiment the method may comprise determining the level of N-acetylglutamate and indole-3-acetate and comparing the levels to control levels. In another embodiment the method may comprise determining the level of N-acetylglutamate and 2-oxoglutarate and comparing the levels to control levels. In another embodiment the method may comprise determining the level of N-acetylglutamate and anserine and comparing the levels to control levels. In another embodiment the method may comprise determining the level of N-acetylglutamate and aspartate. In another embodiment the method may comprise determining the level of N-acetylglutamate and butyrate and comparing the levels to control levels.

In another embodiment the method may comprise determining the level of methionine and acetylcarnitine and comparing the levels to control levels. In another embodiment the method may comprise determining the level of methionine and indole-3-acetate and comparing the levels to control levels. In another embodiment the method may comprise determining the level of methionine and 2-oxoglutarate and comparing the levels to control levels. In another embodiment the method may comprise determining the level of methionine and anserine and comparing the levels to control levels. In another embodiment the method may comprise determining the level of methionine and aspartate. In another embodiment the method may comprise determining the level of methionine and butyrate.

In another embodiment the method may comprise determining the level of indole-3-acetate and 2-oxoglutarate and comparing the levels to control levels. In another embodiment the method may comprise determining the level of indole-3-acetate and anserine and comparing the levels to control levels. In another embodiment the method may comprise determining the level of indole-3-acetate and aspartate. In another embodiment the method may comprise determining the level of indole-3-acetate and butyrate.

In another embodiment the method may comprise determining the level of 2-oxoglutarate and anserine and comparing the levels to control levels. In another embodiment the method may comprise determining the level of 2-oxoglutarate and aspartate. In another embodiment the method may comprise determining the level of 2-oxoglutarate and butyrate.

In another embodiment the method may comprise determining the level of anserine and aspartate. In another embodiment the method may comprise determining the level of anserine and butyrate.

In another embodiment the method may comprise determining the level of aspartate and butyrate.

The method of the invention may comprise determining the level of at least three compounds selected from the group consisting of N-acetylglutamate, methionine, acetylcarnitine, indole-3-acetate, 2-oxoglutarate, anserine, aspartate and butyrate and comparing the levels to control levels. For example, in one embodiment, the method may comprise determining the level of N-acetylglutamate, methionine and acetylcarnitine and comparing the levels to control levels.

The invention includes determining the level of any combination of three compounds selected from the group consisting of N-acetylglutamate, methionine, acetylcarnitine, indole-3-acetate, 2-oxoglutarate, anserine, aspartate and butyrate and comparing the levels to control levels.

The method of the invention may comprise determining the level of at least four compounds selected from the group consisting of N-acetylglutamate, methionine, acetylcarnitine, indole-3-acetate, 2-oxoglutarate, anserine, aspartate and butyrate and comparing the levels to control levels. For example, in one embodiment, the method may comprise determining the level of N-acetylglutamate, methionine, acetylcarnitine and indole-3-acetate and comparing the levels to control levels. In another embodiment, the method may comprise determining the level of N-acetylglutamate, methionine, acetylcarnitine and 2-oxoglutarate and comparing the levels to control levels.

The invention includes determining the level of any combination of four compounds selected from the group consisting of N-acetylglutamate, methionine, acetylcarnitine, indole-3-acetate, 2-oxoglutarate, anserine, aspartate and butyrate and comparing the levels to control levels.

The method of the invention may comprise determining the level of at least five compounds selected from the group consisting of N-acetylglutamate, methionine, acetylcarnitine, indole-3-acetate, 2-oxoglutarate, anserine, aspartate and butyrate and comparing the levels to control levels. For example, in one embodiment, the method may comprise determining the level of N-acetylglutamate, methionine, acetylcarnitine, indole-3-acetate and 2-oxyglutarate and comparing the levels to control levels.

The invention includes determining the level of any combination of five compounds selected from the group consisting of N-acetylglutamate, methionine, acetylcarnitine, indole-3-acetate, 2-oxoglutarate, anserine, aspartate and butyrate and comparing the levels to control levels.

The method of the invention may comprise determining the level of at least six compounds selected from the group consisting of N-acetylglutamate, methionine, acetylcarnitine, indole-3-acetate, 2-oxoglutarate, anserine, aspartate and butyrate and comparing the levels to control levels.

The invention includes determining the level of any combination of six compounds selected from the group consisting of N-acetylglutamate, methionine, acetylcarnitine, indole-3-acetate, 2-oxoglutarate, anserine, aspartate and butyrate and comparing the levels to control levels.

The method of the invention may comprise determining the level of at least seven compounds selected from the group consisting of N-acetylglutamate, methionine, acetylcarnitine, indole-3-acetate, 2-oxoglutarate, anserine, aspartate and butyrate and comparing the levels to control levels.

The invention includes determining the level of any combination of seven compounds selected from the group consisting of N-acetylglutamate, methionine, acetylcarnitine, indole-3-acetate, 2-oxoglutarate, anserine, aspartate and butyrate and comparing the levels to control levels.

The method of the invention may comprise determining the level of N-acetylglutamate, methionine, acetylcarnitine, indole-3-acetate, 2-oxoglutarate, anserine, aspartate and butyrate and comparing the levels to control levels.

Optionally, the method of the invention may comprise determining the level of one or more further markers and comparing the levels to control levels.

The control level in any of the methods discussed above may be determined from a sample from a healthy subject. According to this embodiment, a level of the at least one compound that is increased compared to said control level is indicative of hepatic cancer.

Alternatively, the control level may be determined from a sample from a subject with hepatic cancer. According to this embodiment, a level of the at least one compound that is similar compared to said control level is indicative of hepatic cancer.

Although a sample from a control subject may be assayed in parallel to a sample from a test subject, it may be more convenient to use an absolute control level based on empirical data. An absolute control level provides a threshold such as a threshold level of at least one compound or a threshold profile level. The level of the at least one compound, or the profile level of a sample from a test subject may be compared to the threshold absolute control level wherein a level either higher or lower than the absolute control value is indicative of hepatic cancer.

The levels of the metabolites may be normalised relative to creatinine levels. Surprisingly, the inventors have found that normalisation of levels relative to creatinine provides a useful comparison of test samples and control samples, compared to using the total spectral integral as has been done previously with other markers. Normalisation relative to creatinine corrects for kidney function and muscle wasting that occurs in cancer subjects.

The methods of the invention may be used to test samples from the same subject at two or more different points in time. Performing multiple test on the same subject over time allows the severity of disease to be measured, e.g. to observe whether the disease worsens. Alternatively, multiple testing may allow the efficacy of drugs to be monitored over time.

By N-acetylglutamate is meant a compound with the formula:

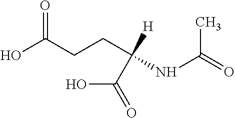

or any naturally occurring variants thereof.

By methionine is meant a compound with the formula:

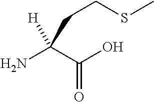

or any naturally occurring variant thereof.

By acetylcarnitine is meant a compound with the formula:

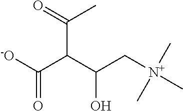

or any naturally occurring variant thereof.

By indole-3-acetate is meant a compound with the formula:

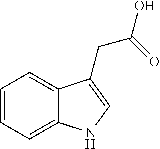 [SMILES: O=C(O)Cc1c[nH]c2ccccc12]

or any naturally occurring variant thereof.

By 2-oxoglutarate is meant a compound with the formula:

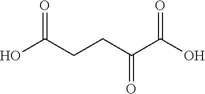

or any naturally occurring variant thereof.

By anserine is meant a compound with the formula:

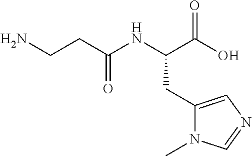 [SMILES: Cn1cncc1C[C@H](NC(=O)CCN)C(=O)O]

or any naturally occurring variant thereof.

By aspartate is meant a compound with the formula:

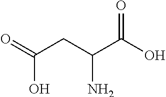

or any naturally occurring variant thereof.

By butyrate is meant a compound with the formula:

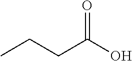

or any naturally occurring variant thereof.

Where any one of the above compounds is ionic, the counter ion may be any ion. Preferably the above compounds are in a neutral form.

The inventors have also found that the levels of N-acetylglutamate, methionine, acetylcarnitine, indole-3-acetate, 2-oxoglutarate, anserine, aspartate and butyrate are significantly increased in samples taken from subjects having more advanced hepatic cancer than subjects having early stage hepatic cancer. Therefore the levels of N-acetylglutamate, methionine, acetylcarnitine, indole-3-acetate, 2-oxoglutarate, anserine, aspartate and butyrate are indicative of the stage of the hepatic cancer. Determining the stage of a hepatic cancer can be useful in determining the prognosis of a patient.

Therefore, the methods of the invention may be used to provide an indication of the stage of a hepatic cancer. The methods of the invention may be used to indicate whether the test subject has stage 1, stage 2 or stage 3 hepatic cancer according to a particular hepatic cancer staging system, e.g. the Okuda staging system. Alternatively, the methods of the invention may be used to more generally to distinguish between subjects having earlier or more advanced hepatic cancer relative to control subjects.

### Hepatic Cancer

The hepatic cancer that may be detected or diagnosed by the methods of the invention may comprise any liver cancer. Hepatic cancer may comprise primary hepatic cancer including but not limited to hepatocellular carcinoma (HCC), fibrolamellar hepatocellular carcinoma, cholangiocarcinoma, angiosarcoma (or haemangiosarcoma) and hepatoblastoma. Alternatively hepatic cancer may comprise secondary hepatic cancer including cancer which has metastasized from the liver to other organs including but not limited to lung, kidney, breast, stomach and colon, skin (e.g. melanoma), prostate, pancreas and cervix.

### Stages of Hepatic Cancer

A number of different systems for assigning the stage of hepatic cancer have been established. These staging systems include but are not limited to Okuda staging, American Joint Committee on Cancer (AJCC) Tumour-Node-Metastasis (TNM); Cancer of Liver Italian Program (CLIP); Barcelona Clinic Liver Cancer (BCLC); Chinese University Prognostic Index (CUPI) and the Japan Integrated Staging (JIS).

Some staging systems such as American Joint Committee on Cancer (AJCC) Tumour-Node-Metastasis (TNM) evaluate only tumour extension. However, in order to usefully give an indication of prognosis, staging of hepatic cancer should include consideration of both tumour extension and liver function.

The Okuda staging system takes into account the proportion of liver tissue that is involved in a tumour, serum concentrations of bilirubin and albumin and the presence or absence of ascites. The Okuda staging system, as with other cancer staging systems assigns patients to stage 1, stage 2 or stage 3 depending on the severity of the parameters listed above. Stage 1 relates to early stage hepatic cancer, stage 2 relates to intermediate stage hepatic cancer and stage 3 relates to advanced hepatic cancer.

Other staging systems such as the CLIP staging system take into account alpha-fetoprotein levels and portal vein thrombosis in addition to tumour distribution. Therefore, by “stage of hepatic cancer” is meant the level of advancement of a hepatic cancer ranging from early stage to advanced stage. The stage of hepatic cancer may be a particular stage number as defined by a particular staging system, such as one of the staging systems described above. Alternatively, the stage of hepatic cancer may refer to a cancer being at an earlier stage or at a more advanced stage relative to another hepatic cancer.

### Subjects

A subject may be any animal e.g., a vertebrate or non-vertebrate animal. Vertebrate animals may be mammals. Vertebrate mammals may be human. Examples of mammals include but are not limited to mouse, rat, pig, dog, cat, rabbit, primate or the like. The subject may be a primate. Preferably the subject is human.

A test subject is a subject from which a sample is obtained and analysed using the methods of the invention. The method of the invention may be performed as a method of diagnosis or prognosis of the test subject. The test subject may be a subject considered to be at risk of hepatic cancer. For example, the test subject may display symptoms of hepatic cancer such as jaundice, bloating from ascites, easy bruising from blood clotting abnormalities, loss of appetite, unintentional weight loss, abdominal pain, especially in the upper-right part, nausea, emesis, and fatigue. Alternatively the test subject may be considered to be at risk of hepatic cancer because they display genetic markers with a known link to hepatic cancer. Alternatively the test subject may be considered to be at risk of hepatic cancer because it tests positive for serum α-fetoprotein. Alternatively, the test subject may be confirmed as suffering from hepatic cancer and the method of the invention may be used to determine the prognosis of the patient and/or the stage of the hepatic cancer.

The methods of the invention may be used for analysing a sample from a subject which is a non-human animal, where the animal is used for screening of drugs for hepatic cancer. The effect of potential drugs on the level of the at least one compound may be indicative of whether the potential drug is efficacious.

The control subject is a subject against which the test subject is compared. The control subject may be a subject with hepatic cancer, in which case, a test subject displaying a similar profile to that of the control profile would be diagnosed as having hepatic cancer. The control subject may be a subject with hepatic cancer of a known stage, in which case, a test subject displaying a similar profile to that of the control profile would be diagnosed as having hepatic cancer of the same stage as the control subject.

Alternatively, the control subject may be a healthy subject, in which case, a test subject displaying a different profile to that of the control subject would be diagnosed as having hepatic cancer. The degree of difference of the profile of a test subject to that of a healthy subject may be indicative of the stage of the hepatic cancer. For example, a test subject displaying an extremely different profile to that of the control subject may be diagnosed as having advanced hepatic cancer. A test subject displaying a significantly different but not extremely different profile to that of the control subject may be diagnosed as having early stage hepatic cancer. Preferably, the control subject is a healthy subject.

A healthy subject may be any subject which does not have hepatic cancer. In a preferred embodiment the method of the invention is able to distinguish between a subject with hepatic cancer and a subject with cirrhosis or a subject with non-cirrhotic liver disease. Subjects with cirrhosis and subjects with non-cirrhotic liver disease are collectively termed subjects with chronic liver disease. By determining the profile of a subject by considering the levels of at least one compound the method of the present invention allows for highly sensitive and specific testing which is able to differentiate between subjects suffering from hepatic cancer and subjects suffering from chronic liver disease, or specifically subjects with cirrhosis or subjects with non-cirrhotic liver disease.

### Sample

The sample tested in the method of the invention may be any biological specimen obtained from a subject. A sample may be a tissue sample. The sample may be obtained with minimal invasiveness or non-invasively, e.g., the sample may be, or may be obtained from blood, plasma, serum, saliva, urine, stool, tears, any other bodily fluid, tissue samples (e.g., biopsy), and cellular extracts thereof (e.g., red blood cellular extract). One skilled in the art will appreciate that samples such as serum samples can be diluted prior to the analysis of levels of compounds.

Preferably the sample is a urine sample. The use of a urine sample in the method of the present invention is particularly advantageous since obtaining the sample from a subject is entirely non-invasive. This is useful where the subject may not wish to undergo invasive procedures in order to be tested for hepatic cancer, e.g. for ethical or religious reasons. Furthermore, the use of urine samples means that samples are obtained in a straightforward manner, and in some embodiments the method of the invention described above or the kit described below may be used to perform self testing.

### Further Compounds

The method of the invention may further comprise determining the level of at least one of the compounds selected from the group consisting of serum α-fetoprotein, creatinine, creatine, carnitine, acetone, glycine, trimethylamine-N-oxide, hippurate, citrate, ribitol, betaphenylpyruvate, 5-hydroxytryptamine, 1-methylnicotinamide, dimethylglycine, N-phenylacetylglycine, betaine aldehyde, 5-hydroxyindole-3-acetate, alpha-hydroxyhippurate, lactate, glutamate, leucine, alanine, choline, phosphorylethanolamine, triglycerides, glucose, glycogen, acetate, N-acetylglycoproteins, pyruvate, glutamine, glycerol, tyrosine, 1-methylhistidine, phenylalanine, low-density lipoprotein, isoleucine, valine, acetoacetate, methyl moieties of fatty acids, methylene moieties of fatty acids, N-acetyl moieties, taurine, unsaturated lipid and very low density lipoproteins or any naturally occurring variants thereof in a sample and comparing the level of the at least one compound to at least one control level.

Measuring further compounds as part of the method of the invention allows the profile of the sample that may be produced to be more accurate, and therefore increases the sensitivity and specificity of the method. The more levels of compounds that are tested, the greater the capability of the method to distinguish subjects which display profiles similar to that of a subject with hepatic cancer but do not suffer from hepatic cancer e.g. cirrhosis sufferers.

In one embodiment, the method of the invention comprises determining the level of creatinine and comparing the level of creatinine with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of creatine and comparing the level of creatine with a control level, wherein the control level is determined from a sample a) from a healthy subject, wherein a level that is increased compared to said control level is indicative of hepatic cancer; and/or b) from a subject with hepatic cancer, wherein a level that is similar compared to said control level is indicative of hepatic cancer.

In one embodiment, the method of the invention comprises determining the level of carnitine and comparing the level of carnitine with a control level, wherein the control level is determined from a sample a) from a healthy subject, wherein a level that is increased compared to said control level is indicative of hepatic cancer; and/or b) from a subject with hepatic cancer, wherein a level that is similar compared to said control level is indicative of hepatic cancer.

In a preferred embodiment, the method of the invention comprises determining the level of N-acetylglutamate, methionine, acetylcarnitine, indole-3-acetate, 2-oxoglutarate, anserine, aspartate, butyrate, creatinine, creatine and carnitine in a sample from a test subject and comparing the level of the compounds to a control level, wherein the control level is determined from a sample from a healthy subject, wherein a level of N-acetylglutamate, methionine, acetylcarnitine, indole-3-acetate, 2-oxoglutarate, anserine, aspartate, butyrate, creatine and/or carnitine that is increased compared to said control level and/or a level of creatinine that is reduced compared to said control level is indicative of hepatic cancer.

In another preferred embodiment, the method of the invention comprises determining the level of N-acetylglutamate, methionine, acetylcarnitine, indole-3-acetate, 2-oxoglutarate, anserine, aspartate, butyrate, creatinine, creatine and carnitine in a sample from a test subject and comparing the level of the compounds to a control level, wherein the control level is determined from a sample from a hepatic cancer patient, wherein a level that is similar to said control level is indicative of hepatic cancer.

In one embodiment, the method of the invention comprises determining the level of glycine and comparing the level of glycine with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of trimethylamine-N-oxide and comparing the level of trimethylamine-N-oxide with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of hippurate and comparing the level of hippurate with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of citrate and comparing the level of citrate with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In a preferred embodiment, the method of the invention comprises determining the level of N-acetylglutamate, methionine, acetylcarnitine, indole-3-acetate, 2-oxoglutarate, anserine, aspartate, butyrate, creatinine, creatine, carnitine, glycine, trimethyl-N-oxide, hippurate and citrate in a sample from a test subject and comparing the level of the compounds to a control level, wherein the control level is determined from a sample from a healthy subject, wherein a level of N-acetylglutamate, methionine, acetylcarnitine, indole-3-acetate, 2-oxoglutarate, anserine, aspartate, butyrate, creatine and/or carnitine that is increased compared to said control level and/or a level of creatinine, glycine, trimethyl-N-oxide, hippurate and/or citrate that is reduced compared to said control level is indicative of hepatic cancer.

In another preferred embodiment, the method of the invention comprises determining the level of N-acetylglutamate, methionine, acetylcarnitine, indole-3-acetate, 2-oxoglutarate, anserine, aspartate, butyrate, creatinine, creatine, carnitine, glycine, trimethyl-N-oxide, hippurate and citrate in a sample from a test subject and comparing the level of the compounds to a control level, wherein the control level is determined from a sample from a hepatic cancer patient, wherein a level that is similar to said control level is indicative of hepatic cancer.

In one embodiment, the method of the invention comprises determining the level of ribitol and comparing the level of ribitol with a control level, wherein the control level is determined from a sample a) from a healthy subject, wherein a level that is increased compared to said control level is indicative of hepatic cancer; and/or b) from a subject with hepatic cancer, wherein a level that is similar compared to said control level is indicative of hepatic cancer.

In one embodiment, the method of the invention comprises determining the level of betaphenylpyruvate and comparing the level of betaphenylpyruvate with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In one embodiment, the method of the invention comprises determining the level of 5-hydroxytryptamine and comparing the level of 5-hydroxytryptamine with a control level, wherein the control level is determined from a sample a) from a healthy subject, wherein a level that is reduced compared to said control level is indicative of hepatic cancer; and/or b) from a subject with hepatic cancer, wherein a level that is similar compared to said control level is indicative of hepatic cancer.

In a preferred embodiment, the method of the invention comprises determining the level of ribitol, betaphenylpyruvate and 5-hydroxytryptamine in a sample from a test subject and comparing the level of the compounds to a control level, wherein the control level is determined from a sample from a healthy subject, wherein a level of ribitol that is increased compared to said control level and/or a level of betaphenylpyruvate and/or 5-hydroxytryptamine that is reduced compared to said control level is indicative of hepatic cancer.

In another preferred embodiment, the method of the invention comprises determining the level of ribitol, betaphenylpyruvate and 5-hydroxytryptamine in a sample from a test subject and comparing the level of the compounds to a control level, wherein the control level is determined from a sample from a hepatic cancer patient, wherein a level that is similar to said control level is indicative of hepatic cancer.

In a preferred embodiment, the method of the invention comprises determining the level of N-acetylglutamate, methionine, acetylcarnitine, indole-3-acetate, 2-oxoglutarate, anserine, aspartate, butyrate, creatinine, creatine, carnitine, glycine, trimethyl-N-oxide, hippurate, citrate, ribitol, betaphenylpyruvate and 5-hydroxytryptamine in a sample from a test subject and comparing the level of the compounds to a control level, wherein the control level is determined from a sample from a healthy subject, wherein a level of N-acetylglutamate, methionine, acetylcarnitine, indole-3-acetate, 2-oxoglutarate, anserine, aspartate, butyrate, creatine, carnitine and/or ribitol that is increased compared to said control level and/or a level of creatinine, glycine, trimethyl-N-oxide, hippurate, citrate, betaphenylpyruvate and/or 5-hydroxytryptamine that is reduced compared to said control level is indicative of hepatic cancer.

In another preferred embodiment, the method of the invention comprises determining the level of N-acetylglutamate, methionine, acetylcarnitine, indole-3-acetate, 2-oxoglutarate, anserine, aspartate, butyrate, creatinine, creatine, carnitine, glycine, trimethyl-N-oxide, hippurate, citrate, ribitol, betaphenylpyruvate and 5-hydroxytryptamine in a sample from a test subject and comparing the level of the compounds to a control level, wherein the control level is determined from a sample from a hepatic cancer patient, wherein a level that is similar to said control level is indicative of hepatic cancer.

In one embodiment, the method of the invention comprises determining the level of at least one further compound selected from the group consisting of acetate, choline, pyruvate, 1-methylnicotinamide, dimethylglycine, N-phenylacetylglycine, betaine aldehyde, 5-hydroxyindole-3-acetate and alpha-hydroxyhippurate and comparing the level of the compounds with a control level, wherein a level of acetate, choline, 1-methylnicotinamide, dimethylglycine, N-phenylacetylglycine and/or betaine aldehyde that is increased compared to said control level and/or a level of pyruvate, 5-hydroxyindole-3-acetate and/or alpha-hydroxyhippurate that is reduced compared to said control level is indicative of hepatic cancer.

In another embodiment, the method of the invention comprises determining the level of acetate, choline, pyruvate, 1-methylnicotinamide, dimethylglycine, N-phenylacetylglycine, betaine aldehyde, 5-hydroxyindole-3-acetate and alpha-hydroxyhippurate in a sample from a test subject and comparing the level of the compounds to a control level, wherein the control level is determined from a sample from a hepatic cancer patient, wherein a level that is similar to said control level is indicative of hepatic cancer.

In a preferred embodiment, the method of the invention comprises determining the level of N-acetylglutamate, methionine, acetylcarnitine, indole-3-acetate, 2-oxoglutarate, anserine, aspartate, butyrate, creatinine, creatine, carnitine, glycine, trimethyl-N-oxide, hippurate, citrate, ribitol, betaphenylpyruvate, 5-hydroxytryptamine, acetate, choline, pyruvate, 1-methylnicotinamide, dimethylglycine, N-phenylacetylglycine, betaine aldehyde, 5-hydroxyindole-3-acetate and alpha-hydroxyhippurate in a sample from a test subject and comparing the level of the compounds to a control level, wherein the control level is determined from a sample from a healthy subject, wherein a level of N-acetylglutamate, methionine, acetylcarnitine, indole-3-acetate, 2-oxoglutarate, anserine, aspartate, butyrate, creatine, carnitine, ribitol, acetate, choline, 1-methylnicotinamide, dimethylglycine, N-phenylacetylglycine and/or betaine aldehyde that is increased compared to said control level and/or a level of creatinine, glycine, trimethyl-N-oxide, hippurate, citrate, betaphenylpyruvate, 5-hydroxytryptamine, pyruvate, 5-hydroxyindole-3-acetate and/or alpha-hydroxyhippurate that is reduced compared to said control level is indicative of hepatic cancer.

In another embodiment, the method of the invention comprises determining the level of N-acetylglutamate, methionine, acetylcarnitine, indole-3-acetate, 2-oxoglutarate, anserine, aspartate, butyrate, creatine, carnitine, glycine, trimethyl-N-oxide, hippurate, citrate, ribitol, betaphenylpyruvate, 5-hydroxytryptamine, acetate, choline, 1-methylnicotinamide, dimethylglycine, N-phenylacetylglycine, betaine aldehyde, 5-hydroxyindole-3-acetate and alpha-hydroxyhippurate in a sample from a test subject and comparing the level of the compounds to a control level, wherein the control level is determined from a sample from a hepatic cancer patient, wherein a level that is similar to said control level is indicative of hepatic cancer.

Herein, by creatinine it is meant a compound with the formula:

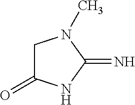

or any naturally occurring variants thereof.

Herein, by creatine, it is meant a compound with the formula:

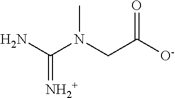

or any naturally occurring variants thereof.

Herein, by carnitine, it is meant a compound with the formula:

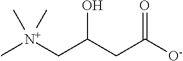

or any naturally occurring variants thereof. In one embodiment carnitine is the L-carnitine enantiomer. Carnitine is a precursor of acetylcarnitine. Therefore, increased levels of carnitine leads to increased levels of acetylcarnitine. Therefore, in some embodiments, an increase in carnitine relative to a control level can be equated to an increase in acetylcarnitine relative to a control level.

By glycine is meant a compound with the formula NH₂CH₂COOH or any naturally occurring variant thereof.

By trimethylamine-N-oxide is meant a compound with the formula (CH₃)₃NO or any naturally occurring variants thereof.

Herein, by hippurate it is meant a compound with the formula:

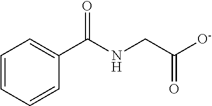 [SMILES: O=C([O-])CNC(=O)c1ccccc1]

or any naturally occurring variants thereof.

Herein, by citrate it is meant a compound with the formula:

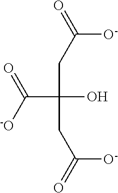

or any naturally occurring variants thereof.

Herein, by ribitol is meant a compound with the formula:

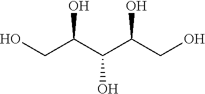

or any naturally occurring variants thereof.

Herein by betaphenylpyruvate is meant a compound with the formula:

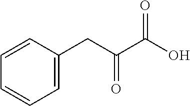 [SMILES: O=C(O)C(=O)Cc1ccccc1]

or any naturally occurring variants thereof.

Herein, by 5-hydroxytryptamine it is meant a compound with the formula:

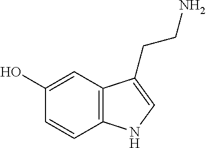 [SMILES: NCCc1c[nH]c2ccc(O)cc12]

or any naturally occurring variants thereof.

Herein, by acetate is meant a compound with the formula CH₃CO₂⁻ or any naturally occurring variant thereof.

Herein, by choline is meant a compound with the formula:

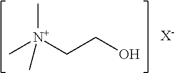

or any naturally occurring variant thereof.

Herein, by 1-methylnicotinamide is meant a compound with the formula:

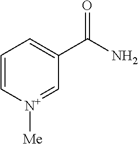

or any naturally occurring variant thereof.

Herein, by dimethylglycine is meant a compound with the formula (CH₃)₂NCH₂COOH or any naturally occurring variant thereof.

Herein, by N-phenylacetylglycine is meant a compound with the formula:

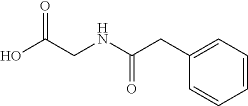 [SMILES: O=C(O)CNC(=O)Cc1ccccc1]

or any naturally occurring variant thereof.

Herein, by betaine aldehyde is meant a compound with the formula:

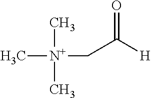

or any naturally occurring variant thereof.

Herein by 5-hydroxyindole-3-acetate is meant a compound with the formula:

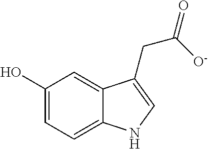 [SMILES: O=C([O-])Cc1c[nH]c2ccc(O)cc12]

or any naturally occurring variant thereof.

Herein, by alpha-hydroxyhippurate is meant a compound with the formula:

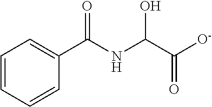 [SMILES: O=C(NC(O)C(=O)[O-])c1ccccc1]

or any naturally occurring variant thereof.

Where any one of the above compounds is ionic, the counter ion may be any ion. Preferably the above compounds are in a neutral form.

### Determining the Level of a Compound

Herein, determining the level of a compound may be achieved using any quantitative or qualitative method known in the art whereby the level of at least one compound from a test sample can be compared to the level of at least one compound from a control sample.

Determination of the at least one level may be achieved by using a single method or a combination of methods.

Determination of the level of a compound may comprise determining a concentration of the compound, or alternatively may comprise determining the level of the compound on a relative scale. The level of a compound that is determined may be normalised relative to the level of another compound. In one embodiment the level of a compound is normalised relative to the level of creatinine, as described above.

Normalisation relative to creatinine takes into account cancer cachexia (muscle wasting that occurs in cancer) and corrects for kidney function.

Methods which may be used to determine the level of the at least one compound may include but are not limited to liquid chromatography, gas chromatography, high performance liquid chromatography (HPLC)¹⁴, ultra high performance liquid chromatography (UPLC), capillary electrophoresis, as well as each of these techniques in combination with mass spectrometry, i.e. liquid chromatography-mass spectrometry¹⁵, gas chromatography-mass spectrometry¹⁶, high performance liquid chromatography-mass spectrometry, capillary electrophoresis-mass spectrometry¹⁷.

Other methods which may be used to determine the level of the at least one compound may include pyrolysis mass spectrometry, refractive index spectroscopy (RI), Ultra-Violet spectroscopy (UV), Near-InfraRed spectroscopy (Near-IR), microwave spectroscopy, Nuclear Magnetic Resonance spectroscopy (NMR)¹⁸, Raman spectroscopy, Light Scattering analysis (LS), thin layer chromatography (TLC), electrochemical analysis, fluorescence analysis, radiochemical analysis, nephelometry, turbidometry, electrical resistance analysis, fluid-solid interaction-based detection, spectrophotometry, colorimetry, optical reflection, heat-of combustion analysis, immunoassays, immunohistochemical assays, and other methods known in the art.

In one embodiment, determination of the level of the at least one compound will be achieved using a spectrophotometric assay. A spectrophotometric assay may be any assay wherein the quantity of a particular compound can be determined by measuring the capacity of a solution containing the substance to absorb light of particular wavelengths. A spectrophotmetric assay may comprise the direct detection of a compound present in a sample, where the compound provides a different absorbance at a known wavelength dependent on the level of compound present. Alternatively, the assay may involve the addition of reagents which undergo a change in absorbance in the presence of a particular compound. This change in absorbance may be measured in order to determine the level of the particular compound of interest.

### Colorimetric Assays

In one embodiment, determination of the level of the at least one compound may be achieved using a colorimetric assay. A colorimetric assay may be any assay in which the level of a compound may be determined by measuring or observing a colour change. A colorimetric assay may be a spectrophotometric assay wherein the wavelength at which the absorbance of the substance is measured is within the visible region of the electromagnetic spectrum. Colorimetric assays may comprise the comparison of the colour of a sample with a colour chart. Colorimetric assays may comprise the addition of reagents that undergo a measurable colour change in the presence of a particular compound.

The determination of the level of each of the at least one compounds may be determined using a separate colorimetric assay. Any colorimetric assay may be used for the determination of the level of a compound wherein the assay causes a colour change that is dependent only on the level of the compound in the sample and is not to a significant degree dependent upon any other variables.

Any assay known in the art may be used to detect the level of at least one compound in a sample.

### Human or Machine Readable Strips

The level of the at least one compound may be determined through use of a human or machine readable strip, in which the level of said at least one compound, may be determined by measuring a change in said human or machine readable strip. A change in the human or machine readable strip may be a change in the human or machine readable strip which occurs via a chemical reaction between a reagent present in or on said human or machine readable strip and said at least one compound. For example, the human or machine readable strip may comprise reagents for performing at least one colorimetric assay to determine the level of at least one compound.

The human or machine readable strip may comprise multiple regions, wherein a separate chemical reaction is conducted in each region. The chemical reaction in each region may be used to detect one of the at least one compounds. On contact with the sample, the reagents present in each region are able to undergo chemical reactions with compounds present in the sample. The levels of each of the at least one compounds may then be determined according to the degree of change, e.g. a colour change that has taken place in each region of the human or machine readable strip.

In one embodiment, where the change in the human or machine readable strip is a colour change, the human or machine readable strip may be read by a human comparing the human or machine readable strip with a chart which shows varying degrees of colour, and which attributes the varying degrees of colour with particular levels of compound.

In another embodiment, the human or machine readable strip may be read by a machine which calculates the degree of change, e.g. a colour change, that has occurred in the human or machine readable strip in each region either by measuring the absorbance of the solution at a particular wavelength or by any other means.

The assays provided in each region of the human or machine readable strip may be any assay which involves a change occurring in a region of the human or machine readable strip wherein the change is dependent only on the level of the at least one compounds.

### Binding Assays

The level of the at least one compound may be determined by a method involving a binding assay. A binding assay may be any assay where one of the at least one compounds is specifically bound by one or more other molecules wherein the one or more other molecules may subsequently be detected. In one embodiment, the one or more other molecules may be one or more proteins. Proteins which specifically bind one of the at least one compounds may subsequently be detected using an antibody specific to the one or more proteins.

In one embodiment, the one or more other molecules may be antibodies which specifically bind to one of the at least one compounds.

An antibody may be a monoclonal or a polyclonal antibody or a fragment thereof.

The level of the at least one compound may be determined using an immunoassay or an immunohistochemical assay. Examples of immunohistochemical assays suitable for use in the method of the present invention include, but are not limited to, immunofluorescence assays such as direct fluorescent antibody assays, indirect fluorescent antibody (IFA) assays, anticomplement immunofluorescence assays, and avidin-biotin immunofluorescence assays. Other types of immunohistochemical assays include immunoperoxidase assays.

The level of the at least one compound may be determined using an antibody which specifically binds to said compound, and said antibody may be detected through colorimetric or radiometric means otherwise known in the art. In one embodiment, the level of the at least one compound may be determined using a sandwich assay, whereby one of the at least one compounds is specifically bound by one protein, and specifically detected by a second protein.

The level of a compound may be determined by providing a binding protein which is known to specifically bind to said compound. Said binding protein may subsequently be detected by an antibody specific to said binding protein.

### Profiling the Sample

A profile of the sample may be determined by analysing the level of the at least one compound. Profiling the sample allows differences between groups of subjects to be characterised by a combination of metabolite ratios (a “metabolic profile”) rather than a single metabolite.

A profile of the sample may be used to determine, on the basis of the levels of the at least one compound, whether the subject has hepatic cancer. A profile of the sample allows for an overall comparison of the levels of compounds in a sample from a test subject and a sample from a control subject.

Profiling may involve normalisation which may include consideration of the level of the at least one compound relative to external compounds, e.g. urinary creatinine, as described above. Normalisation may for example normalise the levels depending on the dilution of the sample by comparing levels of other compounds which are not altered in subjects with hepatic cancer compared to healthy subjects. Alternatively, profiling may involve calculating the ratios of the at least one compounds. Profiling may give more weight to certain compounds of the at least one compounds compared to others.

### Kits

The invention provides a kit for use in diagnosing hepatic cancer and/or determining the stage of hepatic cancer in a subject, the kit comprising at least one reagent for determining the level of a compound selected from the group consisting of N-acetylglutamate, methionine, acetylcarnitine, indole-3-acetate, 2-oxoglutarate, anserine, aspartate and butyrate.

The kit of the invention may comprise at least two reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise at least three reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise at least four reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise at least five reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise at least six reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise at least seven reagents for determining the level of a compound selected from the group consisting of N-acetylglutamate, methionine, acetylcarnitine, indole-3-acetate, 2-oxoglutarate, anserine, aspartate and butyrate.

The kit of the invention may comprise reagents for determining the level of N-acetylglutamate, methionine, acetylcarnitine, indole-3-acetate, 2-oxoglutarate, anserine, aspartate and butyrate.

The kit of the invention may comprise further reagents for determining the level of one or more further compounds selected from the group consisting of creatine, carnitine, glycine, trimethyl-N-oxide, hippurate, citrate, ribitol, betaphenylpyruvate, 5-hydroxytryptamine, acetate, choline, 1-methylnicotinamide, dimethylglycine, N-phenylacetylglycine, betaine aldehyde, 5-hydroxyindole-3-acetate and alpha-hydroxyhippurate.

The reagents used in the kits of the invention for determining the level of the at least one compound may cause a colour change in the assay dependent of the level of the at least one compound.

The reagents used in the kits of the invention for determining the level of the at least one compound may comprise at least one antibody which binds specifically to the at least one compound.

The kits of the invention may further comprise instructions for use.

The kits of the invention may comprise reagents for determining the level of the at least one compound placed on a test strip. Different regions of the test strip may comprise reagents for determining the level of multiple compounds and therefore different assays may be performed in different regions of the test strip.

In one embodiment a kit of the invention may comprise a test strip comprising reagents for determining the level of at least one compound, and a comparison chart.

A comparison chart may shows varying degrees of colour against which the colour present on the test strip can be measured. The comparison chart may attribute the varying degrees of colour which may be present on the test strip with particular levels of compound).

### BRIEF DESCRIPTION OF THE FIGURES

FIG. 1A: Representative spectra from i) Healthy, ii) Non-cirrhosis HBV+, iii) cirrhosis and iv) HCC subjects; showing chemical shift regions (ppm) representative of metabolites. The abbreviations used refer to: Cr:creatinine, Gly:glycine, Indole:indole-3-acetate, Hip:hippurate, Car:carnitine, A-Car:acetylcarnitine, TMAO:trimethylamine-N-oxide, DMA:dimethylamine, 2-oxo:2-oxoglutarate, 4-CP:4-cresolsulphate, NAG:N-acetylglutamate;

FIG. 1B: Principal components analysis (PCA) model coloured by class of subjects from West Africa (Circles: hepatocellular carcinoma; Squares: cirrhosis; Triangles: non-cirrhotic liver disease and Upside down triangles: healthy volunteers.

FIG. 2: Orthogonal partial least squares discriminant analysis (OPLS-DA) loadings plot labelled by metabolites that discriminated HCC subjects from non-cirrhotic liver disease subjects. Metabolites above the horizontal line had higher concentration in HCC subjects than non-cirrhotic liver disease subjects, whereas those below the line were higher in concentration in non-cirrhotic liver disease subjects than HCC subjects. The colour scale highlights the metabolites that are strongly statistically correlated with the disease group. The abbreviations used refer to A-Car: acetylcarnitine; Car: carnitine; 2-Oxo: 2-Oxoglutarate; Cr: creatinine; Hip: hippurate; Gly: glycine; TMAO: trimethylamine N-oxide; DMA: dimethylamine; 4-CP: 4-cresol sulphate; NAG: N-acetylglutamate

FIG. 3: Diagnostic performance measured by area under the characteristic receiver operating curves (AUROC) of urinary proton NMR spectroscopy predictive models (dashed line) compared to serum AFP (solid line) for HCC subjects versus A) healthy subjects B) non-cirrhotic liver disease subjects, C) liver cirrhosis subjects, and D) chronic liver disease subjects (combination of non-cirrhotics and cirrhotics)

FIG. 4. Trends in metabolite concentrations of some identified metabolites by study classes. The graphs compare metabolite levels in HCC subjects; cirrhosis subjects, non-cirrhotic liver disease subjects and healthy subjects.

FIG. 5. Linear regression of relative concentration of metabolites by Okuda stage of HCC (2=combined Okuda stages 1 and 2; 3=Okuda stage 3)

FIG. 6. Linear regression of relative concentration of metabolites by BCLC stage of HCC

FIG. 7. Bar charts showing changes in median concentrations of urinary metabolites among 3 phenotypic states—I=Chronic liver disease control; II=Okuda stages 1 and 2; and III=Okuda stage 3 (Number of * represents the degree of statistical significance compared to control).

FIG. 8. Bar charts showing changes in median concentrations of urinary metabolites among 3 phenotypic states—Chronic liver disease; BCLC A-C; and BCLC D. The abbreviations used refer to AceCar—acetylcarnitine; Bet—betaine aldehyde; Car—carnitine; Crea—creatine; Rib—ribitol; NAG—N-acetylglutamate. FIG. 8A relates to patient set 1, and FIG. 8B relates to patient set 2.

### EXAMPLES

The work leading to this invention has received funding from the European Union Seventh Framework Programme [FP7/2007-2013] under grant agreement n° 265994 (PROLIFICA).

### Example 1

### Patient Selection

### Patient set 1

A total of 290 West African subjects were recruited at study sites in Nigeria (Jos University Teaching Hospital) and Gambia (Medical Research Council, Fajara, Gambia). This total consisted of 63 HCC subjects; 32 cirrhosis (Cir) subjects; 107 non-cirrhotic liver disease subjects (DC) and 88 healthy subjects (NC) The characteristics of the subjects tested are depicted in Table 1 below.

TABLE 1
Clinical and baseline laboratory characteristics of patients and control subjects recruited
at Jos University Teaching Hospital, Nigeria and Medical Research Council, Gambia

| | | | | | Non-cirrhotic | | | |
|---|---|---|---|---|---|---|---|---|
| | | | Cirrhosis | | liver disease | | Healthy | |
| | HCC subjects | | subjects | | subjects | | controls | |
| | (n = 63) | | (n = 32) | | (n = 107) | | (n = 88) | |
| Age; yrs(Median, range) | 46 | (26-80) | 39 | (21-58) | 37 | (22-82) | 41 | (26-98) |
| Male/Female, n (%) | 50/13 | (79.4/20.6) | 25/7 | (78.1/21.9) | 58/49 | (54.2/45.8) | 36/52 | (40.9/59.1) |
| Okuda stage† | | | | | | | | |
| Stage I, n (%) | 2 | (4.3) | / | | / | | / | |
| Stage II, n (%) | 26 | (55.3) | / | | / | | / | |
| Stage III, n (%) | 19 | (40.4) | / | | / | | / | |
| Serum AFP values | 201 | (0-1,085) | 150 | (0.2-853) | 7 | (0.2-902) | 15 | (1.6-387) |
| (median, range); ng/mL | | | | | | | | |
| Serum albumin, 31-43 g/L | 26.0 | (4.0-49.0) | 23.0 | (10.0-47.0) | 41.5 | (4.4-57.0) | 42.0 | (22.0-51.0) |
| Serum ALT, 10-55(M); | 72.0 | (4.0-1,336) | 41.0 | (8.0-122.0) | 28.0 | (2.0-498.0) | 28.0 | (13.0-65.0) |
| 7-30(F) IU/L | | | | | | | | |
| Serum creatinine, | 83.5 | (1.0-273.0) | 85.0 | (5.4-899.0) | 79.0 | (9.2-1000.0) | 70.0 | (46.0-116.0) |
| 59-104 μmol/L | | | | | | | | |
| Serum bilirubin, | 19.2 | (0.7-241.5) | 54.5 | (2.4-884.3) | 11.0 | (0.6-83.4) | 12.0 | (7.0-28.0) |
| 0-17 μmol/L | | | | | | | | |
| Aetiology of liver disease | | | | | | | | |
| HBV, n (%) | 25 | (39.7) | 23 | (71.9) | 103 | (96.3) | / | |
| HCV, n (%) | 12 | (19.0) | 2 | (6.2) | 1 | (0.9) | / | |
| HBV/HCV, n (%) | 2 | (3.2) | 1 | (3.1) | 1 | (0.9) | / | |
| Neg hepatitis, n (%) | 16 | (25.4) | 3 | (9.4) | 0 | (0.0) | 88 | (100) |

| | | | | | | | |
|---|---|---|---|---|---|---|---|
| Unknown, n (%) | 8 | (12.7) | 3 | (9.4) | 2 | (1.9) | / |

†Unable to classify 15subjects owing to insufficient information

Patients with HCC were significantly older than the other classes and comprised mostly of males (79%). About 96% of HCC cases were at Okuda stages II and III at recruitment. HBV constituted the most common aetiological factor for HCC in this study population. 25(39.7%) had HBV, 12(19%) with HCV, whereas a substantial proportion of HCC subjects [16(25.4%)] had neither HBV nor HCV.

### Patient set 2

A further 463 subjects were recruited for validation studies. The characteristics of the subjects tested are depicted in Table 2 below.

TABLE 2
Clinical and baseline laboratory characteristics of patients and control subjects recruited
at Jos University Teaching Hospital, Nigeria and Medical Research Council, Gambia

| | | | | | Non-cirrhotic | | | |
|---|---|---|---|---|---|---|---|---|
| | | | Cirrhosis | | liver disease | | Healthy | |
| | HCC subjects | | subjects | | subjects | | controls | |
| | (n = 141) | | (n = 56) | | (n = 178) | | (n = 88) | |
| Age; yrs(Median, range) | 45 | (21-95) | 36 | (15-69) | 38 | (17-75) | 46 | (18-81) |
| Male/Female, n (%) | 104/31 | (77/23) | 43/13 | (77/23) | 92/85 | (52/48) | 45/43 | (51/49) |
| Aetiology of liver disease | | | | | | | | |
| HBV, n (%) | 63 | (53) | 54 | (98) | 171 | (98) | / | |
| HCV, n (%) | 14 | (12) | 1 | (2) | 2 | (1) | / | |
| HBV/HCV, n (%) | 3 | (3) | 0 | (0) | 0 | (0) | / | |
| Neg hepatitis/unknown, n (%) | 39 | (33) | 0 | (0) | 2 | (1) | 84 | (100) |

### Example 2

### Urine Sample Collection

5 mL of non-fasted urine samples were collected and stored at −80° C. before undergoing air transportation on dry ice. Prior to spectral acquisition, samples were thawed and prepared according to standard methodology¹⁹: 400 μL urine sample was mixed with 200 L of phosphate buffer solution (0.2 M Na₂HPO₄/0.04 M NaH₂PO₄, pH=7.4 plus 0.1% sodium azide, 1 mM 3-trimethylsilyl-1-[2,2,3,3,-²H₄]propionate (TSP)) to stabilize the urinary pH. The samples were allowed to stand for 10 min prior to centrifugation at 13000 rpm for 10 min in order to remove insoluble material. 400 μL of the supernatants from each urine sample was aliquoted into 5 mm NMR tubes (Wilmad LabGlass™, New Jersey, USA) for proton nuclear magnetic resonance (¹H NMR) analysis.

### Example 3

### ¹H NMR Spectroscopy Spectral Acquisition and Processing

Samples were run in a random, non-grouped order. ¹H NMR spectra were acquired using a Bruker Avance 600 MHz NMR spectrometer operating at 600.13 MHz for ¹H at 300 K equipped with a 5 mm broad-band inverse configuration probe. Samples were randomly analysed in automation with a B-ACS 60 sample changer system. Samples were analysed using water suppressed 1D NMR spectrum using the NOESYPRESAT pulse sequence (256 transients)²⁰. Irradiation of the solvent (water) resonance was applied during presaturation delay (2.0 s) for all spectra and for the water suppressed 1D NMR spectra also during the mixing time (0.1 s). The pulse sequence parameters including the 90° pulse (˜10 μs), pulse frequency (˜4.8 ppm), receiver gain (˜200), and pulse powers were optimised for each sample set run. The spectral width was 20 ppm for all spectra.

The NMR data were processed with an exponential line broadening of 1.0 Hz prior to Fourier transformation, which were collected with approximately 32 k real data points. Data [−1.0 to 10.0 ppm] were imported into MATLAB 7.0 software (MathWorks, Natick, Mass.), where they were automatically phased, baseline corrected and referenced to the TSP peak (0.00 ppm), using scripts written in-house. To reduce analytical variation between samples the residual water signal (4.70-5.00 ppm) was truncated from the data set. Normalization to total area was performed by calculating, for each spectrum individually, the ratio between each variable and the sum of each spectrum after removal of regions specified above. Assignment of endogenous urinary metabolites was made by reference to published literature data (²¹²²). This was further confirmed by statistical total correlation spectroscopy (STOCSY), an in-house MATLAB tool (23).

### Example 4

### Multivariate Statistical Analysis

### Method

Differences between patient groups were characterised using a combination of metabolite ratios (a “metabolic profile”) rather than a single metabolite. Multivariate statistical analysis in the form of principal components analysis (PCA) and partial least squared discriminant analysis (PLS-DA) were used for initial analysis²⁴. PCA is an unsupervised analytical tool that provides an overview of complex data through an examination of the covariance structure, highlighting sample outliers and clustering. PLS-DA is a supervised analytical method that relates metabolite data to class membership, elucidating separation between the groups. Supervised orthogonal partial least squares discriminant analysis (OPLS-DA) was performed using software programs (MATLAB 7.0 and SIMCA 13.0). Using these techniques, outliers (due to obvious analytical variation or undiagnosed clinical diabetes) were identified; and some were removed (in order not to influence the model adversely). The supervised models were subjected to “leave-one-out” validation, a technique in which each sample in turn was excluded from the analysis, a model created from the remainder of the samples and the class membership of the excluded sample predicted²⁵. This technique was applied to leave out every 7^{th} sample. Many iterations were performed to determine the predictability required to successfully infer the disease status of the excluded samples.

### Results

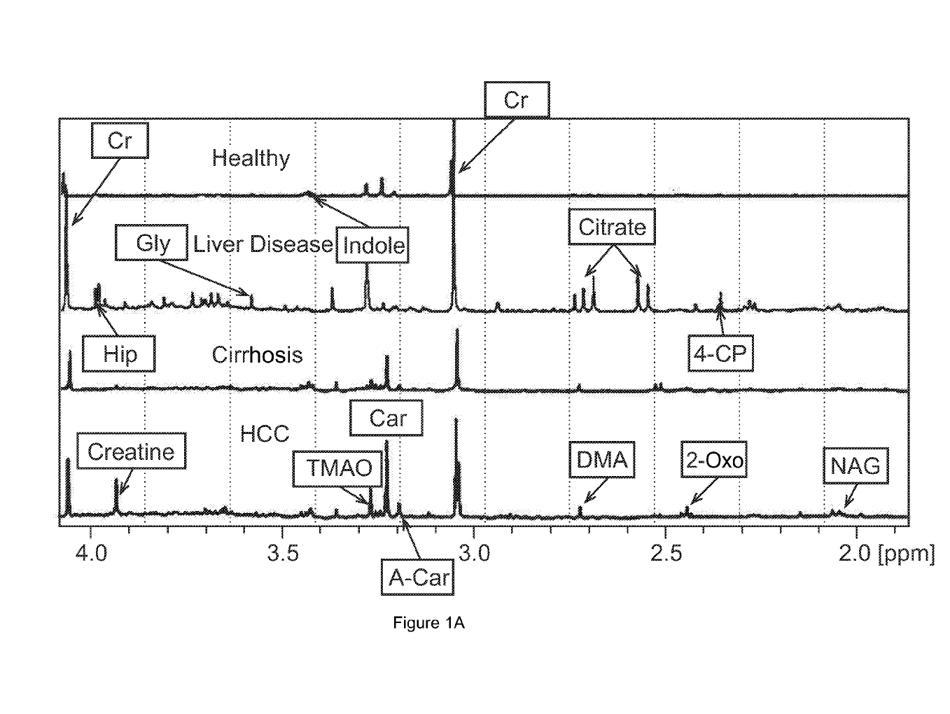

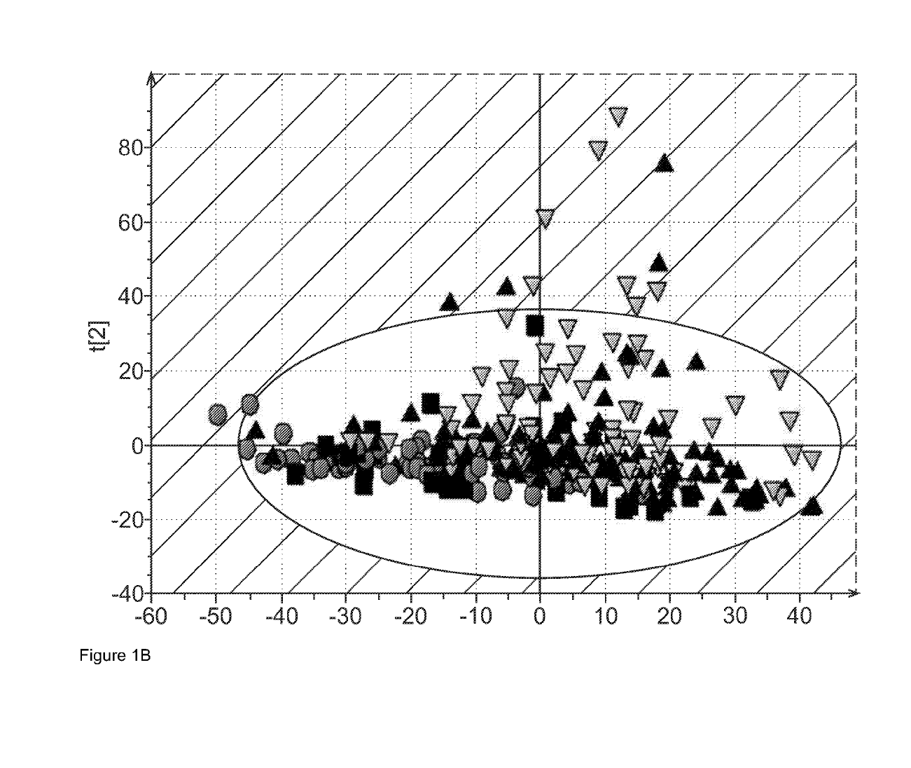

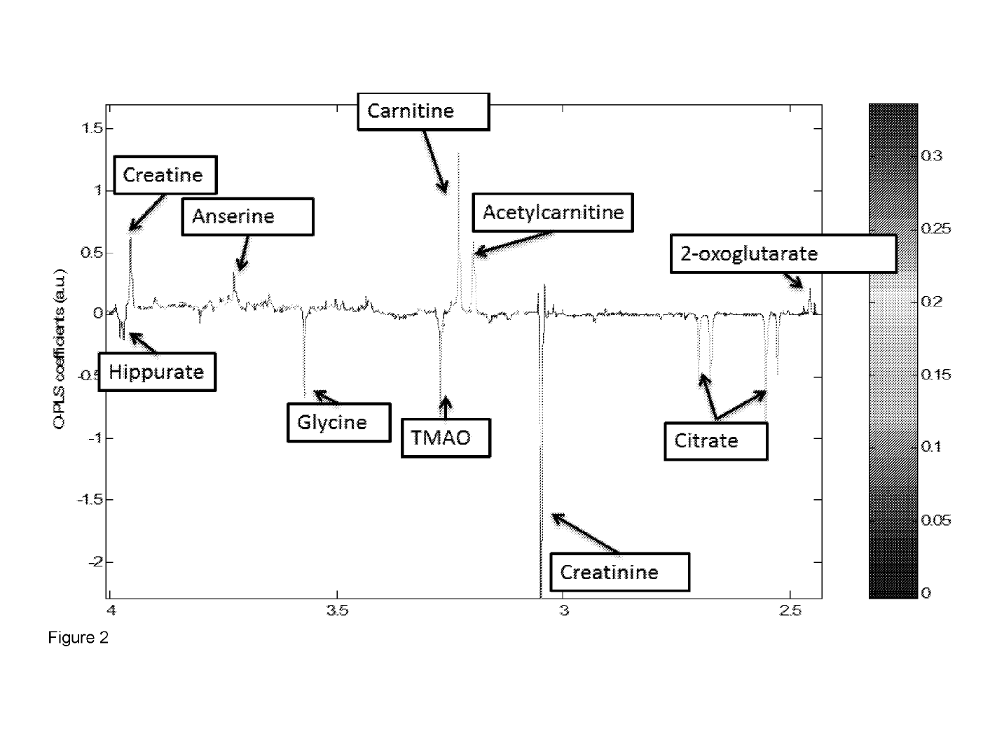

Principal components analysis of samples demonstrated class clustering Representative urinary spectra from the four subject cohorts are displayed in FIG. 1A. Orthogonal partial least squares discriminant analysis of various class combinations, including HCC subjects vs. cirrhosis subjects, HCC subjects vs. non-cirrhotic liver diseases subjects, and HCC subjects vs. healthy subjects identified a number of discriminatory metabolites of HCC as shown in FIG. 2.

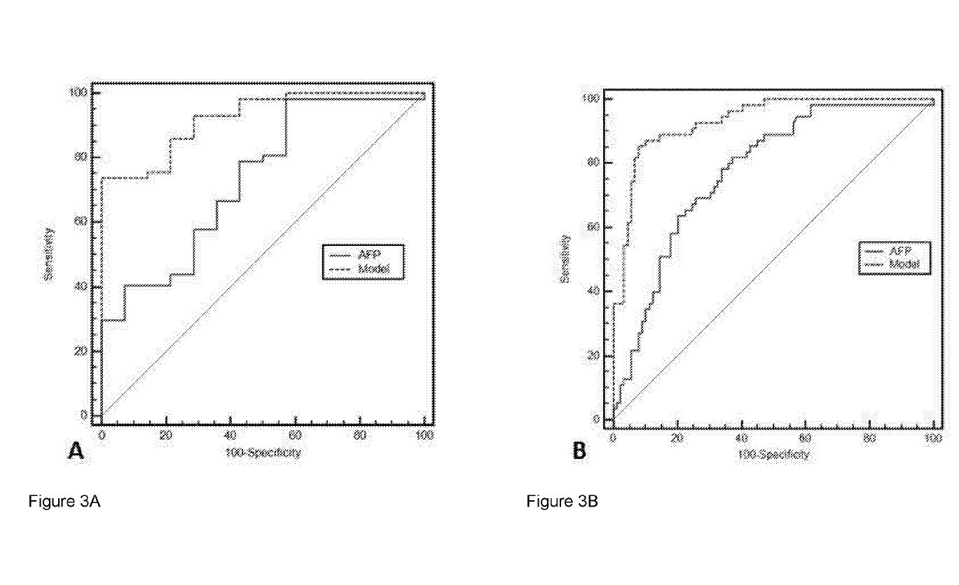

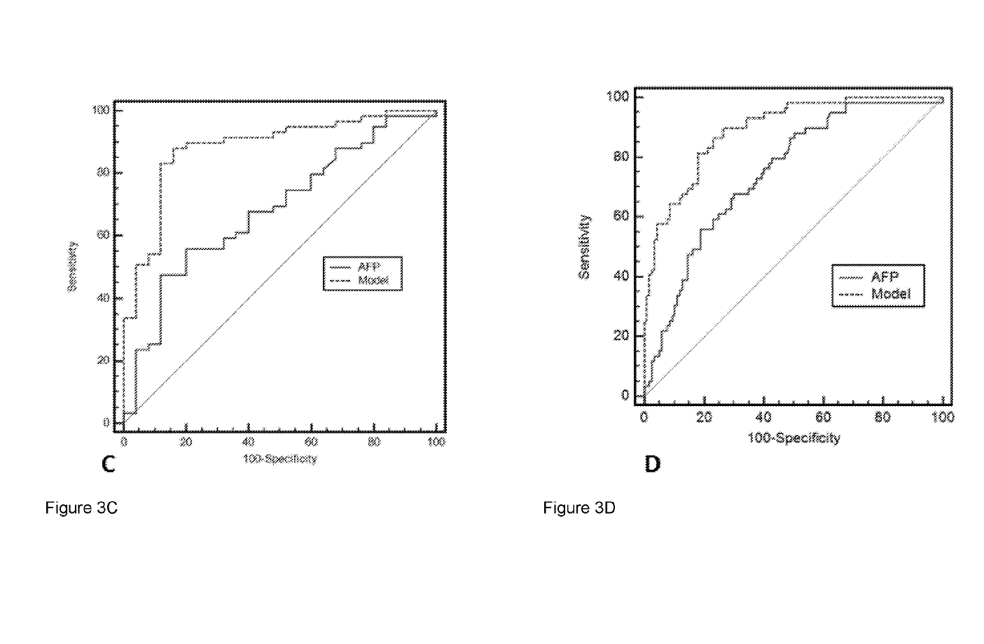

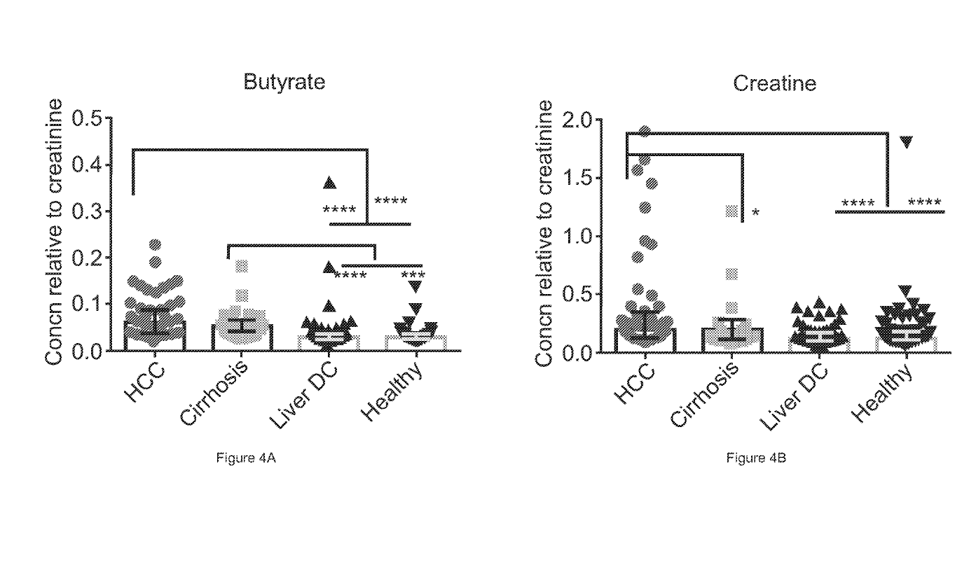

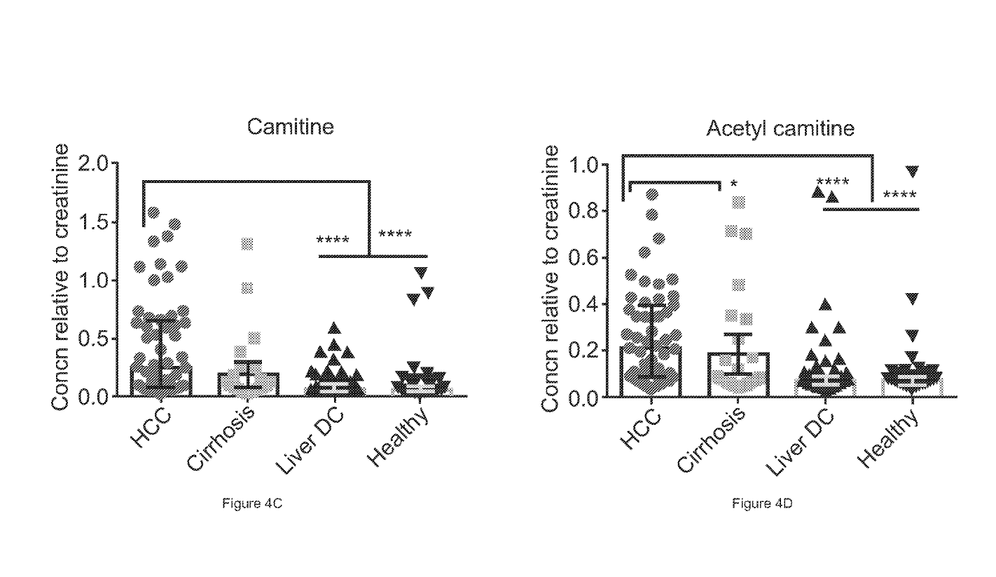

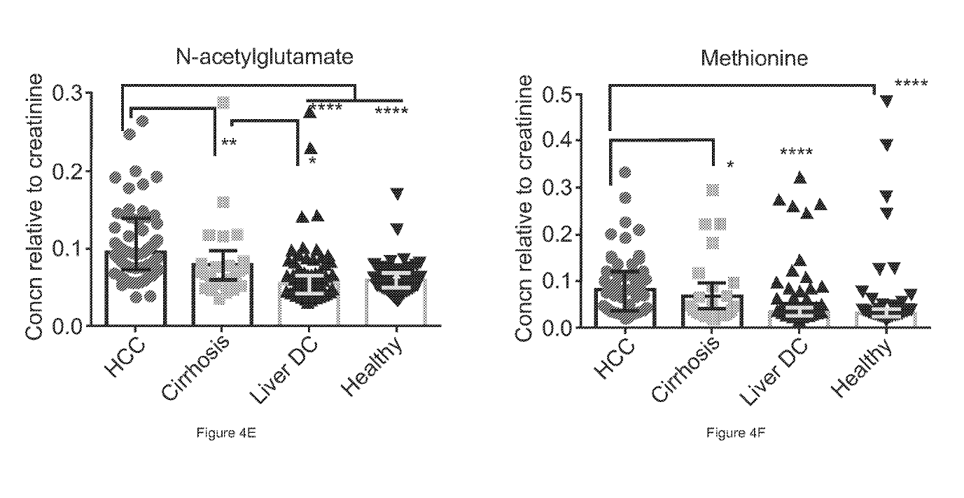

Multivariate modelling of the spectral data showed a distinct profile for urine of HCC subjects compared to cirrhosis subjects, non-cirrhotic liver disease subjects and healthy subjects with both sensitivity and specificity. The sensitivities (95% CI) of the NMR model in discriminating HCC subjects from healthy subjects, non-cirrhotic liver disease subjects and cirrhosis subjects were 97% (89-100), 85% (73-94) and 81% (77-95) respectively, whereas that for AFP were 74% (60-85), 75% (65-85) and 75% (62-85) respectively (see FIG. 3). Similarly, the specificities of NMR model were 99% (94-100), 93% (86-97) and 84% (64-96) for HCC subjects vs. healthy subjects, HCC subjects vs. non-cirrhotic liver disease subjects and HCC subjects vs. cirrhosis subjects respectively. There were comparatively lower specificities of AFP to discriminate HCC from these classes [57% (29-82), 66% (56-76) and 44% (24-65) respectively].

In view of the fact that HCC screening is recommended for HBV-infected sub-Saharan Africans (>20 yrs) and/or those who are known to have established cirrhosis, non-cirrhotic liver disease subjects and cirrhosis subjects were combined into a single class of subjects, chronic liver disease subjects. The AUC of the chronic liver disease metabolite model (0.9) was significantly greater than serum AFP (0.7) in discriminating HCC subjects from chronic liver disease subjects (p=0.0006).

### Example 5

### Univariate Statistical Analysis

### Method

The most important discriminatory metabolites were identified through analysis of the orthogonal partial least squares discriminant analysis (OPLS-DA) loadings plot. Identified metabolites were confirmed using statistical total correlation spectroscopy (STOCSY)²³. Intensities of the metabolites were expressed as the concentration relative to creatinine in order to control for differential renal function of the subjects. Using GraphPad Prism v6 (California, USA), Mann-Whitney U tests were applied to confirm statistical differences in the median values of identified metabolites between HCC subjects and cirrhosis, non-cirrhotic liver disease and healthy subject classes. The ability of each model and single metabolites to discriminate HCC subjects from cirrhosis subjects, non-cirrhotic liver disease subjects and healthy subjects was examined for sensitivity and specificity using Area under the Receiver Operating Characteristic (AUROC) curves. AUROC curves were built to compare the diagnostic performance of these metabolite panels with serum alpha fetoprotein; each for HCC subjects versus cirrhosis subjects, HCC subjects versus non-cirrhotic liver disease subjects and HCC subjects versus healthy subjects. The regression coefficients of each of the significant metabolites were correlated to the Okuda stage of HCC.

### Results

Initially, metabolites corresponding to the resonances that contributed most strongly in discriminating between HCC subjects and the different control groups were identified from the multivariate analysis above. The metabolites that were significantly increased (p<0.0001, except indicated) in HCC subjects compared to all groups of control subjects were N-acetylglutamate, methionine, acetylcarnitine, carnitine, 2-oxoglutarate, indole-3-acetaldehyde, and creatine; whereas creatinine was significantly lower in urine of HCC than controls (see Table 3). Citrate, 4-cresol sulphate (p=0.0006) and trimethylamine N-oxide were additional metabolites found to be significantly lower in the urine of HCC subjects compared to healthy subjects. Anserine was higher in urine of HCC subjects compared to healthy subjects. Urinary metabolite panel performed better than AFP in discriminating HCC from other non-HCC liver conditions.

TABLE 3
Median fold change (FC) of urinary metabolites
in HCC subjects, compared to controls

| | HCC vs. | HCC vs. | HCC vs. | HCC vs. |
|---|---|---|---|---|
| Metabolite | Healthy | DC | Cirrhosis | CLD |
| Acetylcarnitine | 3.0**** | 3.2**** | 3.2*** | 3.3**** |
| Alanine | 1.4**** | 1.4*** | | 1.3*** |
| Anserine | 2.4**** | 2.1**** | 1.5** | 1.9**** |
| Arginine | 1.3*** | | | |
| Aspartate | | 2.2**** | | 2.0**** |
| Butyrate | | 2.1**** | | 2.0**** |
| Carnitine | 4.6**** | 3.9**** | 3.0** | 3.9**** |
| Citrate | 2.5**** | 1.1 | | |
| Creatine | 1.7**** | 1.7**** | 1.8*** | 1.9**** |
| Creatinine | −1.4**** | −1.4**** | −1.5*** | −1.4**** |
| 4 Cresol sulphate | −1.5** | −1.3 | | |
| Dimethylamine | 1.3** | 1.3**** | | 1.3**** |
| Hippurate | −1.3* | −1.1 | −1.4** | |
| Histidine | | 1.2*** | | 1.1* |
| Indole-3-acetate | 1.6**** | 2.2**** | 1.4*** | 2.5**** |
| Lysine | 1.6*** | 1.5*** | | |
| Methionine | 2.8**** | 2.6**** | 2.5*** | 2.6**** |
| N-acetylglutamate | 1.7**** | 1.7**** | 1.6**** | 1.7**** |
| 2 Oxoglutarate | 2.2**** | 1.9**** | 1.9*** | 2.0**** |
| Trimethylamine N-oxide | −1.5**** | −1.1 | | −1.1 |

*indicates degree of significant difference (* = 0.0, ** = 0.00, *** = 0.000 and **** = 0.0000) and positive value indicates higher FC in HCC than controls while negative value indicates reverse increase in controls than HCC)

The diagnostic performance of a number of the urinary metabolites were comparable or improved relative to serum AFP, as shown in Table 4 below.

TABLE 4
Sensitivity and specificity of diagnosis
using different individual metabolites.

| Metabolite | Sensitivity (%) | Specificity (%) |
|---|---|---|
| Serum AFP | 47.5 (34.3-60.9) | 80.0 (59.3-93.2) |
| Acetylcarnitine | 74.6 (61.6-85.0) | 72.0 (50.6-87.9) |
| Creatine | 81.4 (69.1-90.3) | 64.0 (42.5-82.0) |
| Methionine | 61.0 (47.4-73.5) | 80.0 (59.3-93.2) |
| N-acetylglutamate | 72.9 (59.7-83.6) | 80.0 (59.3-93.2) |

The fact that individual urinary metabolites can provide comparable or improved diagnostic performance relative to serum AFP shows that using a panel of these markers to provide a metabolic profile provides an even further improved method of diagnosis.

### Example 6

### Correlation of Metabolites with Stage of HCC

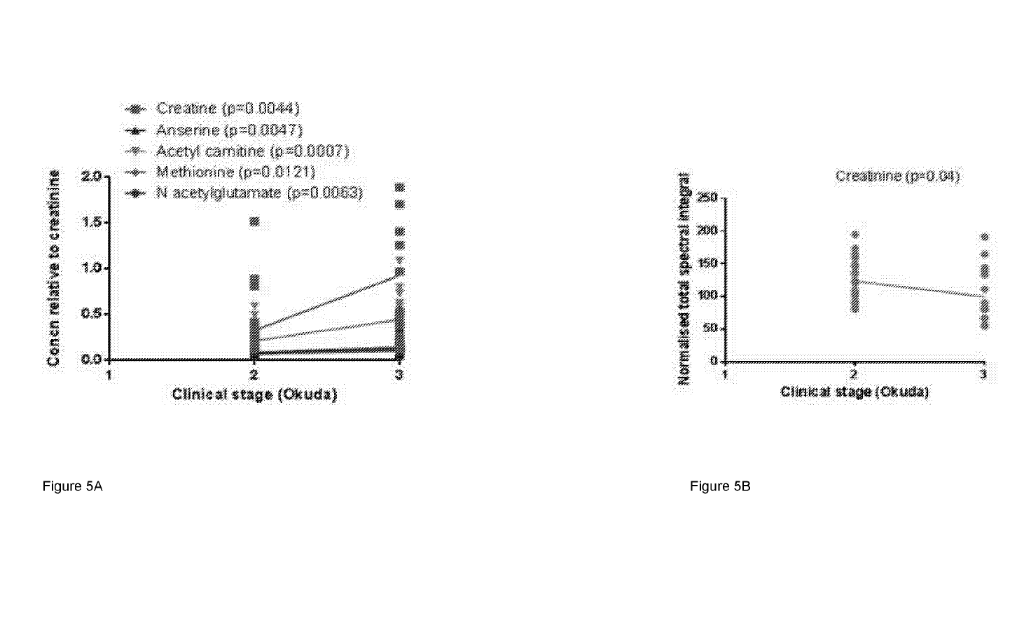

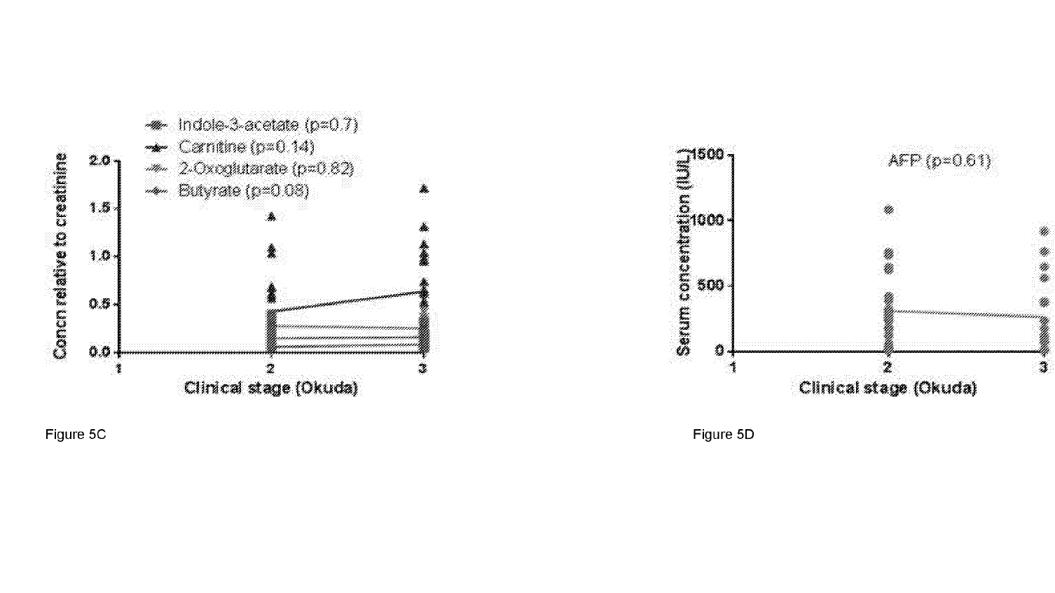

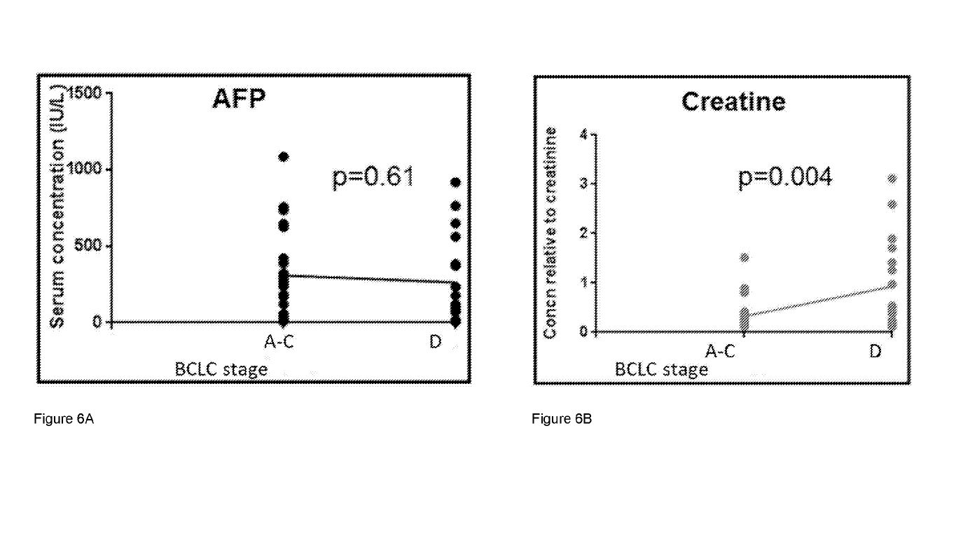

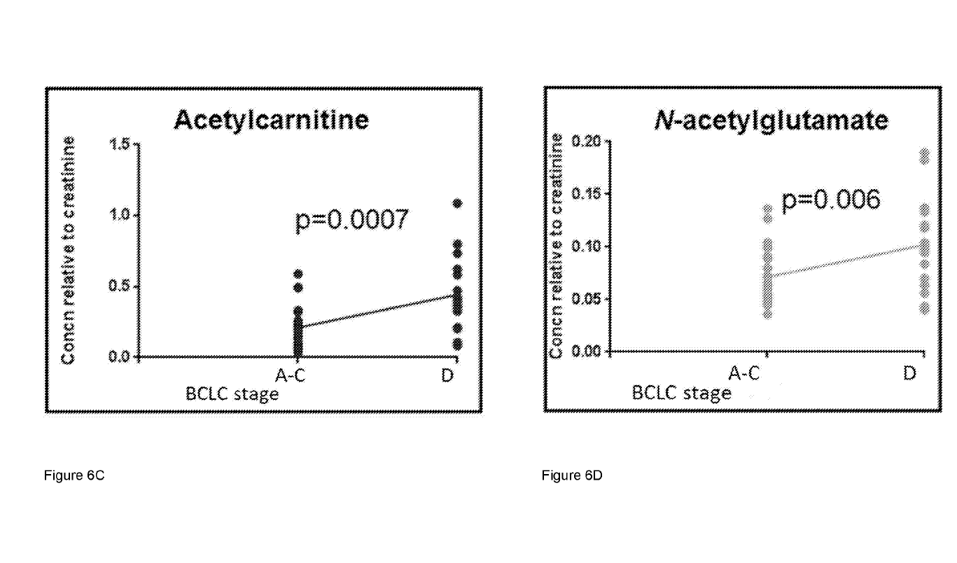

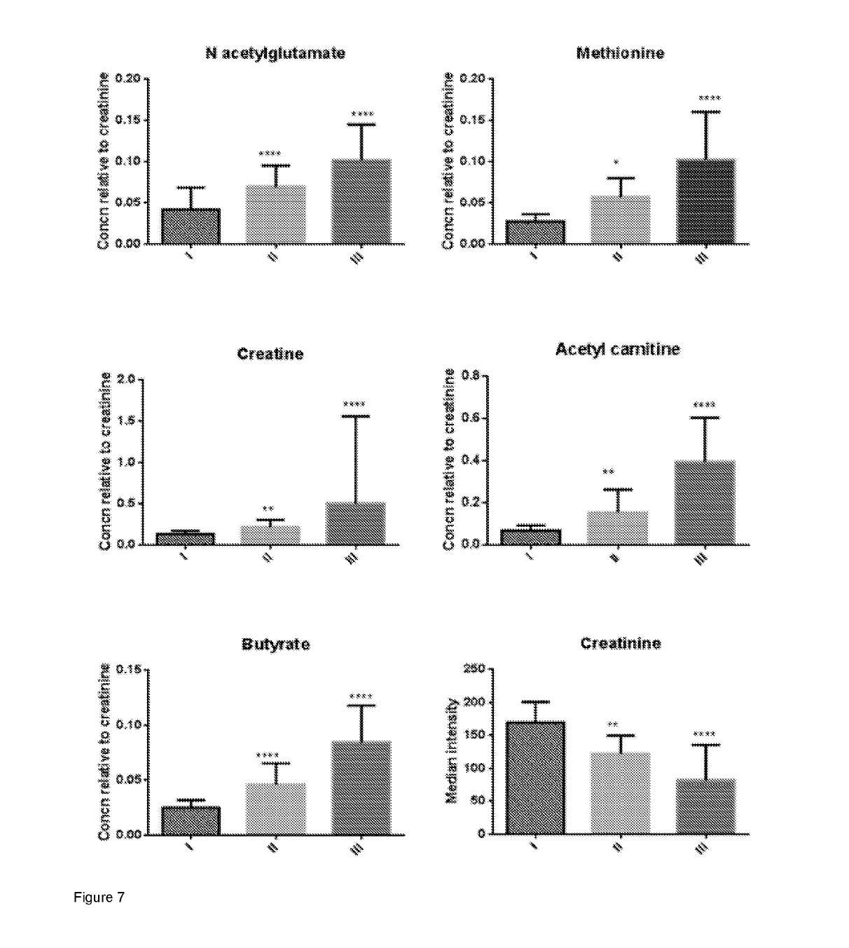

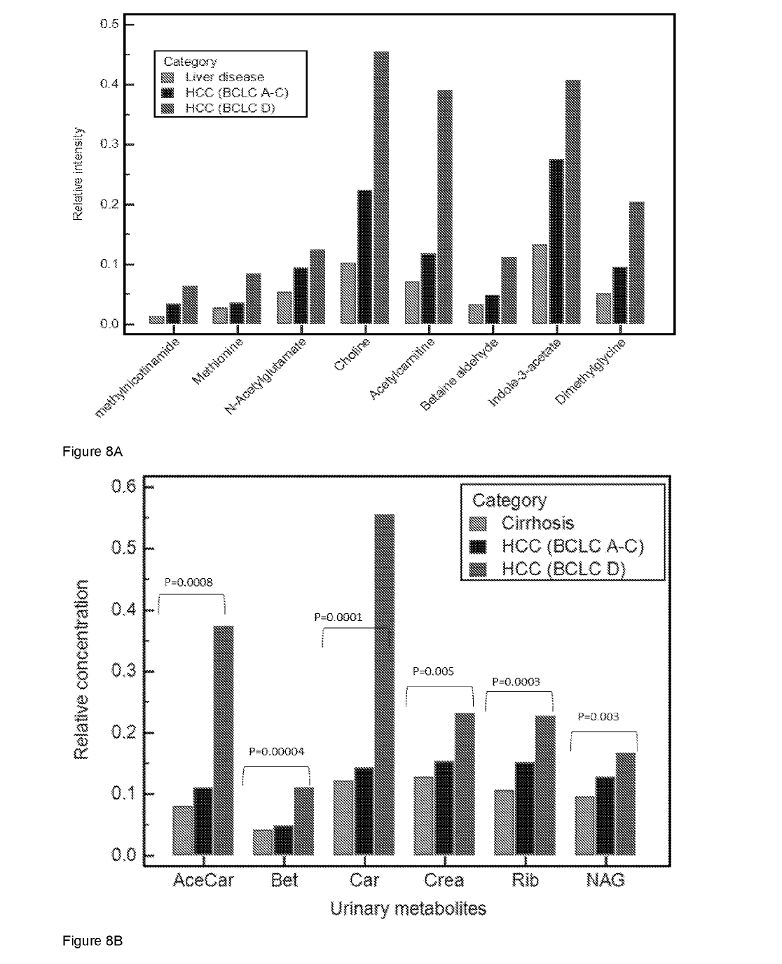

There were only two HCC subjects used in the study that were diagnosed with Okuda stage 1 at recruitment. These subjects were therefore included these with stage 2 subjects for statistical considerations. There was poor correlation with Okuda stage of HCC using serum AFP (p=0.61); as well as by some of the urinary metabolites that had good diagnostic ability, such as indole-3-acetate (p=0.38), 2-oxoglutarate (p=0.82) and carnitine (p=0.08). However, the relative concentration of N-acetylglutamate, methionine, acetylcarnitine, butyrate, aspartate, anserine, and creatine were significantly positively correlated to clinical stage of HCC, whereas creatinine was negatively correlated (see FIG. 5). The relative concentrations of creatine, acetylcarnitine and N-acetylglutamate (but not AFP) have also been shown to be positively correlated with HCC stage when the HCC stage of the subjects tested is assessed using the BCLC staging method (see FIG. 6). Separating patients into three categories; chronic liver disease subjects, Okuda stages 1 & 2; and Okuda stage 3, significant trends were observed for many metabolites (see FIG. 7). Similarly, separating patients into three categories based on the BCLC staging method showed significant trends for many metabolites in both of the patient sets tested (see FIG. 8).

### REFERENCES

¹El-Serag, H. B. Hepatol. Res. (Suppl. 2) S88-S94
²Taylor-Robinson, S. D. et al., Lancet 1997, 350 (9085), 1142-1143
³Khan, S. A, et al., J. Hepatol. 2002, 37 (6), 806-813
⁴Shariff M I et al., Expert Rev Gastroenterol Hepatol. 2009, 3 (4), 353-367
⁵www.hepatocellular.org
⁶Chien-Jen Chen et al., JAMA 2006, 295 (1): pp. 65-73.
⁷Furui J, Furukawa M, Kanematsu T. The low positive rate of serum alpha-fetoprotein levels in hepatitis C virus antibody-positive patients with hepatocellular carcinoma. Hepatogastroenterology 1995 September; 42(5):445-449.
⁸Nguyen M H, Keeffe E B. Screening for hepatocellular carcinoma. J Clin Gastroenterol 2002 November; 35(5 Suppl 2):S86-S91.
⁹Peng Y C, Chan C S, Chen G H. The effectiveness of serum alpha-fetoprotein level in anti-HCV positive patients for screening hepatocellular carcinoma. Hepatogastroenterology 1999 November; 46(30):3208-3211.
¹⁰Stefaniuk P, Cianciara J, Wiercinska-Drapalo A. Present and future possibilities for early diagnosis of hepatocellular carcinoma. World J Gastroenterol 2010 Jan. 28; 16(4):418-424.
¹¹Raedle, J. et al., C. Dig. Dis. Sci 1995, 40 (12), 2587-2594.
¹²Gomaa et al., World J. Gastroenterol, in press.
¹³El-Serag H B, Marrero J A, Rudolph L, Reddy K R (May 2008). “Diagnosis and treatment of hepatocellular carcinoma”. Gastroenterology 134 (6): 1752-63.
¹⁴Yang J., et al. J. Chromoatogra B, 2004, 813, 1-2, 59-65
¹⁵Lu, X. et al., J. Chromatogra. B, 2008, 866, 64
¹⁶Pasikanti, K. K. et al., J. Chromatagr. B, 2008, 871, 202
¹⁷Benavente F. et al., Electrophoresis, 2006, 27, 4570
¹⁸Hollywood, K. et al., Proteomics 6, 2006, 4716
¹⁹Beckonert O, Keun H C, Ebbels T M, Bundy J, Holmes E, Lindon J C, et al. Metabolic profiling, metabolomic and metabonomic procedures for NMR spectroscopy of urine, plasma, serum and tissue extracts. Nat Protoc 2007; 2(11):2692-2703.
²⁰Ross A, Schlotterbeck G, Klaus W, Senn H. Automation of NMR measurements and data evaluation for systematically screening interactions of small molecules with target proteins. J Biomol NMR 2000 Feb.; 16(2):139-146.
²¹Bollard M E, Keun H C, Beckonert O, Ebbels T M, Antti H, Nicholls A W, et al. Comparative metabonomics of differential hydrazine toxicity in the rat and mouse. Toxicol Appl Pharmacol 2005 Apr. 15; 204(2):135-151.
²²Nicholson J K, Foxall P J, Spraul M, Farrant R D, Lindon J C. 750 MHz 1H and 1H-13C NMR spectroscopy of human blood plasma. Anal Chem 1995 Mar. 1; 67(5):793-811.
²³Cloarec O, Dumas M E, Craig A, Barton R H, Trygg J, Hudson J, et al. Statistical total correlation spectroscopy: an exploratory approach for latent biomarker identification from metabolic 1H NMR data sets. Anal Chem 2005 Mar. 1; 77(5):1282-1289.
²⁴Trygg J, Holmes E, Lundstedt T. Chemometrics in metabonomics. J Proteome Res 2007 Feb.; 6(2):469-479.
²⁵Kohavi, Ron. A study of cross-validation and bootstrap for accuracy estimation and model selection. 2 ed. 1995. 1137-1143.

1. A method for analysing a sample from a test subject comprising:
i) determining the level of at least one compound selected from the group consisting of N-acetylglutamate, methionine, acetylcarnitine, indole-3-acetate, 2-oxoglutarate, anserine, aspartate and butyrate in the sample from the test subject; and
ii) comparing the level of the at least one compound determined in step i) to at least one control level, wherein the levels of the at least one compound are indicative of whether the subject has hepatic cancer.

2. The method of claim 1, wherein the control level is determined from a sample from a healthy subject, wherein a level that is increased compared to said control level is indicative of hepatic cancer.

3. The method of claim 1, wherein the control level is determined from a sample from a hepatic cancer patient, wherein a level that is similar to said control level is indicative of hepatic cancer.

4. The method of claim 1, comprising determining the level of at least two compounds selected from the group consisting of N-acetylglutamate, methionine, acetylcarnitine, indole-3-acetate, 2-oxoglutarate, anserine, aspartate and butyrate in a sample.

5. The method of claim 1, comprising determining the level of at least three compounds selected from the group consisting of N-acetylglutamate, methionine, acetylcarnitine, indole-3-acetate, 2-oxoglutarate, anserine, aspartate and butyrate in a sample.

6. The method of claim 1, comprising determining the level of at least four, at least five, at least six or at least seven compounds selected from the group consisting of N-acetylglutamate, methionine, acetylcarnitine, indole-3-acetate, 2-oxoglutarate, anserine, aspartate and butyrate in a sample.

7. The method of claim 1, comprising determining the level of N-acetylglutamate, methionine, acetylcarnitine, indole-3-acetate, 2-oxoglutarate, anserine, aspartate and butyrate in a sample.

8. The method of claim 1, wherein the levels of the compounds are normalised relative to levels of creatinine.

9. The method of claim 1, comprising determining a profile for the sample from the test subject using the level of the at least one compound; and comparing the profile of the sample from the test subject with a control profile determined using at least one control level determined from a sample from a control subject.

10. The method of claim 1, further comprising
a) determining the level of at least one further compound selected from the group consisting of ribitol, betaphenylpyruvate and 5-hydroxytryptamine; and
b) comparing the level of the at least one compound determined in step a) to a control level determined from a sample from a healthy subject, wherein
i) an increase in the level of ribitol; and/or
ii) a reduction in the level of betaphenylpyruvate and/or 5-hydroxytryptamine is indicative of hepatic cancer.

11. The method of claim 1, further comprising
a) determining the level of at least one further compound selected from the group consisting of creatinine, creatine and carnitine; and
b) comparing the level of the at least one compound determined in step a) to a control level determined from a sample from a healthy subject, wherein
i) a reduction in the level of creatinine; and/or
ii) an increase in the level of creatine and/or carnitine is indicative of hepatic cancer.

12. The method of claim 1, further comprising
a) determining the level of at least one further compound selected from the group consisting of glycine, trimethylamine-N-oxide, hippurate and citrate; and
b) comparing the level of the at least one compound determined in step a) to a control level determined from a sample from a healthy subject, wherein a reduction in the level of glycine, trimethylamine-N-oxide, hippurate and citrate is indicative of hepatic cancer.

13. The method of claim 1, wherein the levels of the at least one compound are indicative of the stage of hepatic cancer of the test subject.

14. The method of claim 13, wherein the control level is determined from a sample from a subject having early stage hepatic cancer, wherein a level that is increased compared to said control level is indicative of advanced hepatic cancer.

15. The method of claim 13, wherein the control level is determined from a sample from a subject having advanced hepatic cancer, wherein a level that is similar to said control level is indicative of advanced hepatic cancer.

16. The method of claim 1, wherein the sample is obtained from a mammal.

17. The method of claim 16, wherein the mammal is a human.

18. The method of claim 1, wherein the sample is selected blood, blood plasma, blood serum, cerebrospinal fluid, bile acid, saliva, synovial fluid, pleural fluid, pericardial fluid, peritoneal fluid, feces, nasal fluid, ocular fluid, intracellular fluid, intercellular fluid, lymph fluid, and urine.

19. The method of claim 18 wherein the sample is urine.

20. The method of claim 1, wherein the hepatic cancer is hepatocellular carcinoma.

21. The method of claim 1, wherein determining the level of a compound comprises contacting the sample with an antibody which binds specifically to said compound.

22. The method of claim 1, wherein determining the level of a compound comprises performing a colorimetric or spectrometric assay on the sample.

23. The method of claim 1, wherein the method is able to distinguish between a patient with hepatic cancer and a patient with cirrhosis.

24. A kit for use in diagnosing hepatic cancer and/or determining the stage of hepatic cancer in a subject, the kit comprising at least one reagent for determining the level of a compound selected from the group consisting of N-acetylglutamate, methionine, acetylcarnitine, indole-3-acetate, 2-oxoglutarate, anserine, aspartate and butyrate.

25. The kit of claim 24, comprising at least two reagents for determining the level of a compound selected from the group consisting of N-acetylglutamate, methionine, acetylcarnitine, indole-3-acetate, 2-oxoglutarate, anserine, aspartate and butyrate.

26. The kit of claim 24, comprising at least three reagents for determining the level of a compound selected from the group consisting of N-acetylglutamate, methionine, acetylcarnitine, indole-3-acetate, 2-oxoglutarate, anserine, aspartate and butyrate.

27. The kit of claim 24, comprising at least four, at least five, at least six, or at least seven reagents for determining the level of a compound selected from the group consisting of N-acetylglutamate, methionine, acetylcarnitine, indole-3-acetate, 2-oxoglutarate, anserine, aspartate and butyrate.

28. The kit of claim 24, comprising reagents for determining the level of N-acetylglutamate, methionine, acetylcarnitine, indole-3-acetate, 2-oxoglutarate, anserine, aspartate and butyrate.

29. The kit of claim 24, wherein the reagents for determining the level of the at least one compound cause a colour change in the assay dependent of the level of the at least one compound.

30. The kit of claim 24, wherein the reagents for determining the level of the at least one compound comprise at least one antibody which binds specifically to the at least one compound.
